(19) United States
(12) Patent Application Publication
(10) Pub. No.: US 2006/0247537 A1
(43) Pub. Date: Nov. 2, 2006

(54) Endoscope apparatus

(76) Inventor: Shinya Matsumoto, Tokyo-to (JP)

Correspondence Address: KENYON & KENYON LLP, 1500 K STREET N.W., SUITE 700, WASHINGTON, DC, 20005, US

(21) Appl. No.: 11/405,554

(22) Filed: Apr. 18, 2006

(30) Foreign Application Priority Data
Apr. 19, 2005 (JP) 2005-120895

(51) Int. Cl. A61B 6/00

(52) U.S. Cl. 600/478

(57) ABSTRACT

A fluorescence endoscope apparatus includes a light source section emitting excitation light for exciting a fluorescent substance, an excitation light supply section irradiating a living body with light emitted from the light source section, and a fluorescence detection section provided with an imaging optical system for imaging fluorescent light emanating from the living body. The light source section has an excitation wavelength selective device selecting a plurality of kinds of excitation light in different wavelength regions and the imaging optical system has an excitation wavelength cutoff filter blocking light of excitation wavelength selected by the excitation wavelength selective means so that the fluorescence detection section detects the number of kinds of fluorescent light in different wavelength region, identical with the number of kinds of excitation light in different wavelength regions, irradiated through the excitation light supply section.

This application claims benefits of Japanese Application No. 2005-120895 filed in Japan on Apr. 19, 2005, the contents of which are hereby incorporated by reference.

### BACKGROUND OF THE INVENTION

1. Field of the Invention

This invention relates to a fluorescence endoscope apparatus in which a living body is irradiated with excitation light and fluorescent light emanating from the living body is imaged to obtain a fluorescent image, and more specifically, to a fluorescence endoscope apparatus in which the diagnosis of a lesion produced in the living body can be made under endoscopy.

2. Description of Related Art

Endoscope apparatuses for making the diagnosis of the lesion produced in the living body are widely known. For example, fluorescence endoscope apparatuses in which the surface of a living tissue is irradiated with excitation light and a fluorescent substance contained in the living tissue is excited so that a fluorescent image is acquired by imaging fluorescent light emanating from the living tissue are put to practical use. The fluorescence endoscope apparatus is provided for the purpose of making the diagnosis of the lesion produced in the living tissue on the basis of information contained in the acquired fluorescent image.

In the case where the surface of the living tissue is irradiated with the excitation light and auto-fluorescence from the surface of the living tissue is detected, it is known that a normal tissue and a lesion tissue are different in the intensity of the auto-fluorescence. Thus, the intensity distribution of fluorescent light derived from the auto-fluorescence image of the living tissue is analyzed, and thereby the area of the lesion tissue can be distinguished from that of the normal tissue. The living tissue assumes a layer structure, and a submucosa of the layer structure contains many collagens and elastins from which the auto-fluorescence is emitted.

When a structural change by the lesion is produced in the tissue of a mucosa layer located above the submucosa, the auto-fluorescence from collagen and elastin undergoes a serious influence of this change and is attenuated until it reaches the surface of the mucosa layer. Consequently, by detecting fluorescence intensities in the range of wavelengths of 420-600 nm that are principal auto-fluorescence wavelengths of collagen and elastin, information for distinguishing the area of the lesion tissue produced in the mucosa layer can be acquired.

On the other hand, porphyrin that is an organic compound existing in the living body is known to have a tendency to accumulation in a tumor. Since porphyrin, like collagen and elastin, emits fluorescent light having a peak wavelength at about 630 nm with excitation light in a wavelength region from blue to green, fluorescence intensities in an extremely narrow region of wavelengths including 630 nm are detected, and thereby information showing that the tumor is produced in the living tissue can be acquired.

Even when a fluorescent medicine, such as 5 ALA (5-aminolevulinic acid), is administered from the outside of the body, porphyrin can be accumulated in the tumor. In this way, auto-fluorescence spectra from the living tissue are separately detected in accordance with the wavelength region, and thereby different information contained in individual spectral regions can be taken out.

A method and apparatus using the auto-fluorescence of the living tissue to make the diagnosis of the living tissue are disclosed, for example, in U.S. Pat. No. 5,769,792. A fluorescence endoscope apparatus disclosed here is such that, by using fluorescent images in the spectral region that the auto-fluorescence intensity of the lesion tissue is substantially different from that of the normal tissue and in the spectral region that the auto-fluorescence intensity of the lesion tissue is substantially equal to that of the normal tissue and in the spectral region, the area of the lesion tissue is clearly displayed. Whereby, the lesion tissue can be sharply distinguished from the normal tissue surrounding it.

A technique of diagnosing the possible lesion tissue in the living tissue by using a substance that has an affinity for the lesion tissue produced in the living body and absorbs excitation light to emit fluorescent light is known. In this case, a fluorescent substance is first administered from the outside of the living body to a part that the existence of the lesion is doubted. After a time, the fluorescent substance is selectively connected to the lesion tissue, and thus the part is then irradiated with the excitation light to detect fluorescent light from the fluorescent substance. In this way, the area of the lesion tissue produced in the living body can be clarified. As such fluorescent substances, fluorescent probes disclosed in PCT Publication Nos. WO 03/079015 and WO 2004/005917 are known.

The fluorescent probe includes a section that a substance (hereinafter called a substance to be detected) peculiarly participating in the process that the lesion tissue, such as the tumor, is produced and grows is captured and connected at its molecule level and a section of pigment emitting fluorescent light. The pigment emitting the fluorescent light can be chosen from varied pigment having been commercially available.

For example, Publication No. WO 03/079015 discloses a fluorescent probe constructed of pigment commercially available, having an excitation wavelength peak and a fluorescence wavelength peak in the region of wavelengths between 600 and 1200 nm. In such a fluorescent probe, its fabrication at a relatively low cost is possible and a verification of safety in the living body is already advanced.

On the other hand, Publication No. WO 2004/005917 discloses a fluorescent probe having the property that before the substance to be detected is captured, the emission of fluorescent light is suppressed and after the substance to be detected is captured, it is changed to a substance emitting substantially strong light. Such a fluorescent probe has the advantage that the accuracy of detection of the lesion can be improved because only when it is connected with the substance to be detected, fluorescent light is emitted. Moreover, since the fluorescent probe can be designed so that a particular substance to be detected is selectively captured and connected, a substance participating in the feature inherent in the lesion is chosen as a target and thereby it is possible to serve the analysis and diagnosis of the peculiarity of the lesion (for example, whether it is cancer).

In the case of the diagnosis of the lesion produced in the living tissue under endoscopy, when much information respecting the lesion contained in an image acquired through the endoscope is obtained, the accuracy of the diagnosis can be improved accordingly. It is thus desirable that the fluorescence endoscope apparatus is provided with both the function of acquiring the auto-fluorescence image from the lesion tissue in the visible wavelength region and the function of acquiring the fluorescence image from the fluorescent prove connected with the lesion tissue in a red-to-near-infrared wavelength region. It is also desirable to have the function of using information respecting the lesion contained in each image to process the image into an image useful for the diagnosis.

On the other hand, in the case where screening in the living body is performed under endoscopy in order to find the lesion produced in the living tissue, it is desirable that information such that the position of the lesion can be accurately specified in an observation field, rather than varied information as to the lesion, is acquired and such information is used so that an image useful for screening in the living body can be provided.

In conventional fluorescence endoscope apparatuses, however, it is impossible to achieve the acquirement of compound information and processing of the image such as those described above.

### SUMMARY OF THE INVENTION

The fluorescence endoscope apparatus according to the present invention comprises a light source section emitting excitation light for exciting a fluorescent substance, an excitation light supply section irradiating a living body with light emitted from the light source section, and a fluorescence detection section provided with an imaging optical system for imaging fluorescent light emanating from the living body. In this case, the light source section has an excitation wavelength selective means selecting a plurality of kinds of excitation light in different wavelength regions and the imaging optical system has an excitation wavelength cutoff filter blocking light of excitation wavelength selected by the excitation wavelength selective means so that the fluorescence detection section detects the number of kinds of fluorescent light in different wavelength region, identical with the number of kinds of excitation light in different wavelength regions, irradiated through the excitation light supply section.

In the fluorescence endoscope apparatus according to the present invention, it is desirable that the light source section is constructed so that excitation light for an auto-fluorescence substance exciting the auto-fluorescence substance existing originally in the living body and excitation light for an administration fluorescent substance different in wavelength from the excitation light for the auto-fluorescence substance exciting a fluorescent substance administered inside the living body from the exterior are emitted through the excitation wavelength selective means.

In the fluorescence endoscope apparatus according to the present invention, the excitation light supply section is preferably constructed so that a plurality of fluorescent substances administered inside the living body from the exterior through the excitation wavelength selective means are excited in different wavelength regions.

In the fluorescence endoscope apparatus according to the present invention, the light source section is preferably constructed so that a plurality of kinds of fluorescent light in different wavelength regions, emitted from the light source section, have wavelengths more than 600 nm in a near infrared region.

In the fluorescence endoscope apparatus according to the present invention, the fluorescence detection section is preferably constructed so that fluorescent light in a predetermined wavelength region between adjacent excitation wavelength regions is detected with respect to a plurality of kinds of excitation light in different wavelength regions, emitted from the light source section.

In the fluorescence endoscope apparatus according to the present invention, the fluorescence detection section is provided at the tip of a scope.

In the fluorescence endoscope apparatus according to the present invention, it is desirable that the fluorescence detection section has a single image sensor.

In the fluorescence endoscope apparatus according to the present invention, it is desirable that the excitation light supply section is constructed so that the living body is irradiated with a plurality of kinds of excitation light in different wavelength regions, each in different timing.

In the fluorescence endoscope apparatus according to the present invention, it is desirable that the image sensor of the fluorescence detection section includes a monochrome CCD.

In the fluorescence endoscope apparatus according to the present invention, it is desirable that the image sensor of the fluorescence detection section includes a color mosaic CCD.

In the fluorescence endoscope apparatus according to the present invention, it is desirable that the light source section is provided with a light source for observations irradiating the living body with white light in different timing, together with a plurality of kinds of excitation light in different wavelength regions.

In the fluorescence endoscope apparatus according to the present invention, it is desirable that the excitation wavelength cutoff filter has the characteristic of transmitting at least a part of light in a visible wavelength region.

In the fluorescence endoscope apparatus according to the present invention, it is desirable to have at least two kinds of image modes for observations and diagnoses of a screening mode and a minute investigation mode in which fluorescence detections are different in number.

These and other features and advantages of the present invention will become apparent from the following detailed description of the preferred embodiments when taken in conjunction with the accompanying drawings.

### BRIEF DESCRIPTION OF THE DRAWINGS

FIG. 1 is a block diagram showing the entire structure of the endoscope apparatus of Embodiment 1 in the present invention;

FIG. 2 is a view showing the structure of an excitation wavelength selective filter used in the endoscope apparatus of Embodiment 1;

FIGS. 3A, 3B, and 3C are graphs showing spectral transmittance characteristics of a first excitation wavelength selective filter, a second excitation wavelength selective filter, and a visible light filter, respectively, used in the endoscope apparatus of Embodiment 1;

FIG. 4 is a graph showing the spectral transmittance characteristic of an excitation wavelength cutoff filter placed in an objective optical system used in the endoscope apparatus of Embodiment 1;

FIG. 5 is a graph showing schematically a wavelength characteristic relationship between excitation light and fluorescent light, used in the endoscope apparatus of Embodiment 1;

FIGS. 6A, 6B, 6C, and 6D are graphs showing timing of light for acquiring images of a screening mode in the endoscope apparatus of Embodiment I with respect to light emitted from a light source device, reflected light from a living body, fluorescent light therefrom, and light incident on an image sensor, respectively;

FIGS. 7A, 7B, 7C, and 7D are graphs showing timing of light for acquiring images of a minute investigation mode in the endoscope apparatus of Embodiment 1 with respect to light emitted from a light source device, reflected light from a living body, fluorescent light therefrom, and light incident on the image sensor, respectively;

FIG. 8 is a view showing the structure of a modified example of the excitation wavelength selective filter used in the endoscope apparatus of Embodiment 1;

FIG. 9 is a block diagram showing the entire structure of a modified example of the endoscope apparatus of Embodiment 1 in the present invention;

FIG. 10 is a block diagram showing the entire structure of another modified example of the endoscope apparatus of Embodiment 1 in the present invention;

FIGS. 11A, 11B, and 11C are graphs showing spectral transmittance characteristics of the first excitation wavelength selective filter, the second excitation wavelength selective filter, and the visible light filter, respectively, used in the endoscope apparatus of Embodiment 2;

FIG. 12 is a graph showing the spectral transmittance characteristics of the excitation wavelength cutoff filter placed in the objective optical system used in the endoscope apparatus of Embodiment 2;

FIGS. 13A and 13B are graphs showing spectral transmittance characteristics of the first excitation wavelength selective filter and the visible light filter, respectively, used in the endoscope apparatus of Embodiment 3;

FIG. 14 is a graph showing the spectral transmittance characteristic of the excitation wavelength cutoff filter placed in the objective optical system used in the endoscope apparatus of Embodiment 3;

FIG. 15 is a graph showing schematically a wavelength characteristic relationship between excitation light and fluorescent light, used in the endoscope apparatus of Embodiment 3;

FIG. 16 is a graph showing absorption spectra and fluorescence spectra relative to organic pigment, Alexa Fluor 555, Alexa Fluor 647, Alexa Fluor 680, and Alexa Fluor 750;

FIG. 17 is a view showing the structure of a common fluorescence endoscope apparatus;

FIG. 18 is a graph showing fluorescence spectra mainly deriving from collagen excited in the vicinity of 400 nm in a normal tissue and a tumor; and

FIG. 19 is a block diagram showing the entire structure of the endoscope apparatus of Embodiment 4 in the present invention.

### DETAILED DESCRIPTION OF THE PREFERRED EMBODIMENTS

Subsequently, the embodiments of the endoscope apparatus of the present invention will be explained.

### Embodiment 1

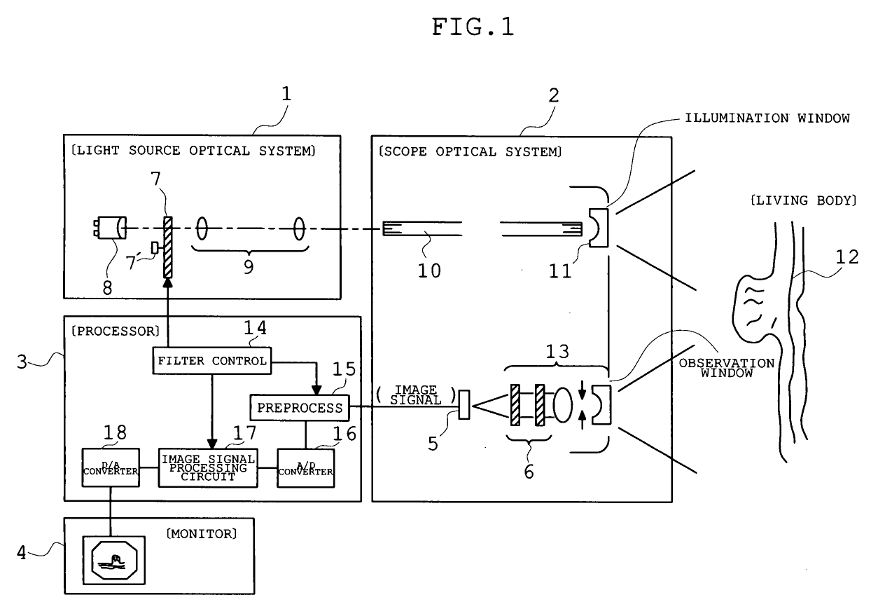

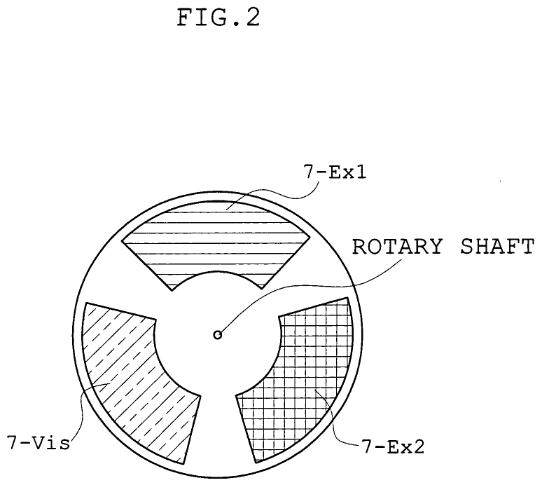

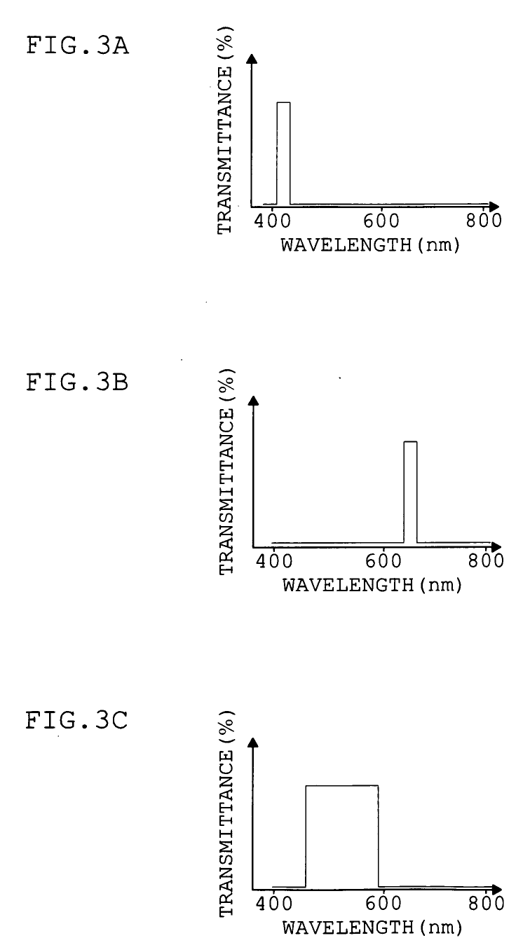

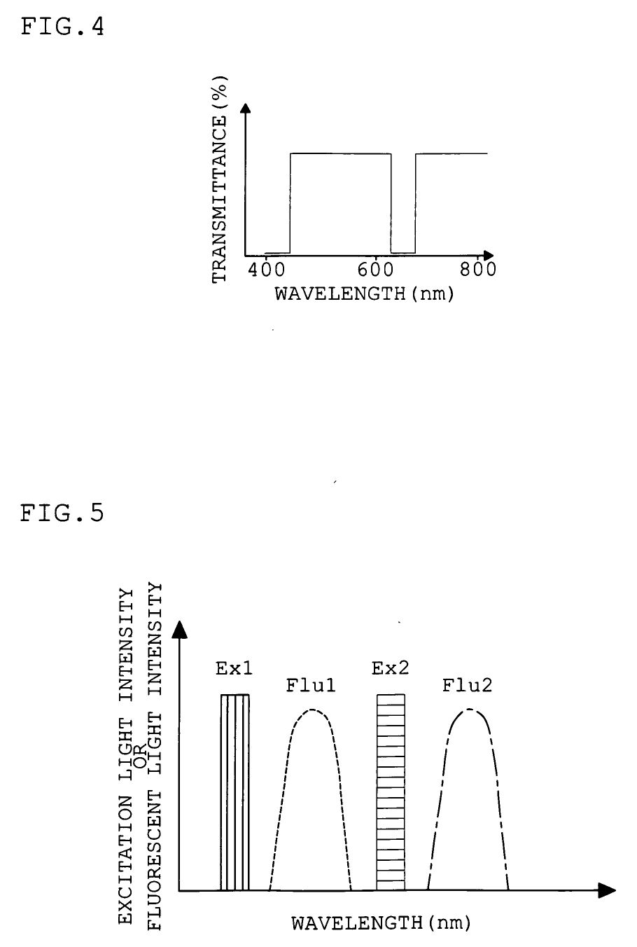

FIG. 1 is a block diagram showing the entire structure of the endoscope apparatus of Embodiment 1 in the present invention; FIG. 2 is a view showing the structure of an excitation wavelength selective filter used in the endoscope apparatus of Embodiment 1; FIGS. 3A, 3B, and 3C are graphs showing spectral transmittance characteristics of a first excitation wavelength selective filter, a second excitation wavelength selective filter, and a visible light filter, respectively, used in the endoscope apparatus of Embodiment 1; FIG. 4 is a graph showing the spectral transmittance characteristic of an excitation wavelength cutoff filter placed in an objective optical system used in the endoscope apparatus of Embodiment 1; and FIG. 5 is a graph showing schematically a wavelength characteristic relationship between excitation light and fluorescent light, used in the endoscope apparatus of Embodiment 1.

The fluorescence endoscope apparatus of this embodiment is constructed so that two modes can be set: a screening mode applied to the case where screening inside the living body is performed under endoscopy in order to find the lesion produced in the living tissue and a minute investigation mode applied to the case where the diagnosis of the lesion produced in the living tissue is made under endoscopy.

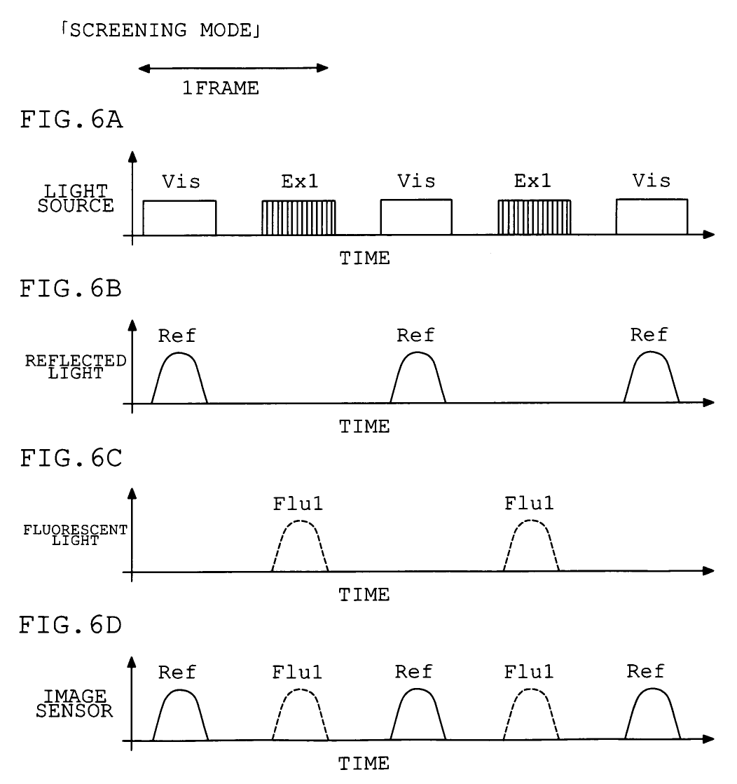

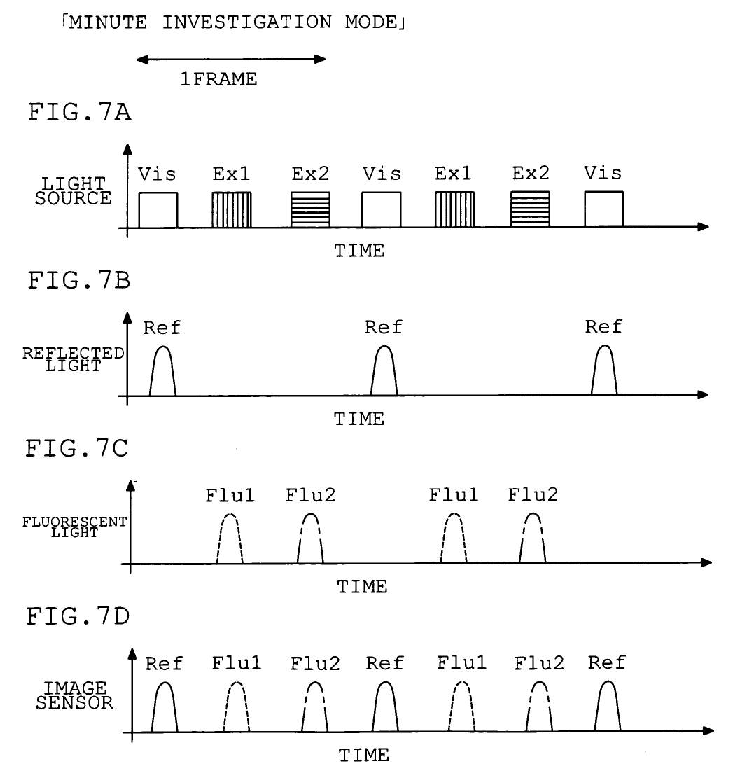

FIGS. 6A, 6B, 6C, and 6D are graphs showing timing of light for acquiring images of the screening mode in the endoscope apparatus of Embodiment 1 with respect to light emitted from a light source device, reflected light from a living body, fluorescent light therefrom, and light incident on an image sensor, respectively. FIGS. 7A, 7B, 7C, and 7D are graphs showing timing of light for acquiring images of the minute investigation mode in the endoscope apparatus of Embodiment 1 with respect to light emitted from a light source device, reflected light from a living body, fluorescent light therefrom, and light incident on the image sensor, respectively.

The fluorescence endoscope apparatus of Embodiment 1, as shown in FIG. 1, comprises a light source device 1, a scope body 2, a processor 3, and a monitor device 4. The light source device 1 as the light source section has a lamp 8 radiating light ranging from a visible region to a near infrared wavelength region, an excitation wavelength selective filter 7 as the excitation wavelength selective means, and a condenser lens 9 and is constructed to emit excitation light of a plurality of wavelengths in a time series.

The scope body 2 has a light guide fiber 10, an illumination lens 11, an objective optical system 13 as the imaging optical system, and an image sensor 5. The objective optical system 13 has an excitation wavelength cutoff filter 6 blocking excitation light that is emitted from the light source device 1 and is made incident by reflection from a living body 12.

The light source device 1, the light guide fiber 10, and the illumination lens 11 constitute the excitation light supply section of the present invention, and the objective optical system 13 and the image sensor 5 constitute the fluorescence detection section of the present invention. The processor 3 is constructed so that the switching of selection of the excitation wavelength by the excitation wavelength selective filter 7 and the timing of the image acquirement are controlled and image processing is performed. The monitor device 4 is constructed so that an image image-processed through the processor 3 is displayed.

The excitation wavelength selective filter 7, as shown in FIG. 2, is such that filters 7-Ex1, 7-Ex2, and 7-Vis, each having a different spectral transmittance characteristic, are arranged in three openings provided in the circumferential direction of a light-blocking disk. The filter 7 is rotated about its rotary shaft by the drive of a motor 7' shown in FIG. 1 and a desired filer, of the filters 7-Ex1, 7-Ex2, and 7-Vis, is set on the optical path so that light with a predetermined wavelength can be selectively transmitted. The spectral transmittance characteristics of the filters 7-Ex1, 7-Ex2, and 7-Vis provided on the excitation wavelength selective filter 7 are as shown in FIGS. 3A, 3B, and 3C, respectively, and light in any of wavelength regions is transmitted. The first excitation wavelength selective filter 7-Ex1 excites the auto-fluorescence substance, for example, of collagen that emits the auto-fluorescence deriving from the living body and thus, as shown in FIG. 3A, has the transmittance characteristic of transmitting wavelengths in the vicinity of 400 nm. The second excitation wavelength selective filter 7-Ex2 excites Alexa 680, prepared by Invitrogen Corporation, which is a fluorescent medicine emitting fluorescent light in the near infrared region and thus, as shown in FIG. 3B, has the transmittance characteristic of transmitting wavelengths in the vicinity of 680 nm. The visible light filter 7-Vis acquires the aspect information of the living body from reflected light from the living body to reflect it in fluorescence information and thus, as shown in FIG. 3C, has the transmittance characteristic of transmitting wavelengths ranging from about 400 nm to about 600 nm in the visible region. Also, the fluorescent medicine Alexa 680 is changed to a probe such as that having a tumor affinity and is previously administered to the living body.

Light emitted from the lamp 8 is such that only light component of a desired excitation wavelength is extracted through the excitation wavelength selective filter 7. The light, after being supplied to a light guide connecter, not shown, of the scope body 2 through the condenser lens 9, is transmitted by the light guide fiber 10 and emerges on the side of the part 12 to be examined in the living body through the illumination lens 11 attached to an illumination window that is provided at the tip of an insertion tube section (namely, the distal end of the scope body 2). The part 12 to be examined is illuminated with light in a selected wavelength region to excite the fluorescent substance accumulated in the living body.

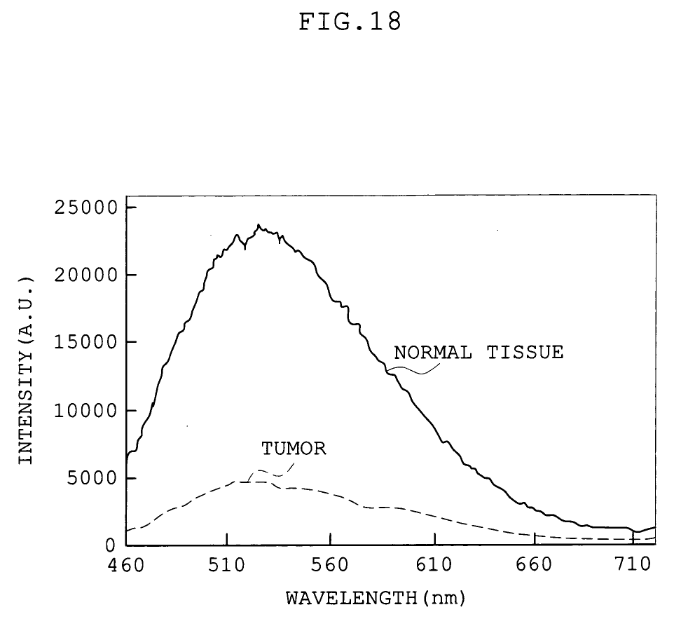

FIG. 5 is a graph showing wavelength regions of first excitation light Ex1, fluorescent light Flu1 by the auto-fluorescence substance of collagen excited by the first excitation light Ex1, second excitation light Ex2, and fluorescent light Flu2 by Alexa 680 excited by the second excitation light Ex2. Also, in FIG. 18, fluorescence spectra mainly deriving from collagen excited in the vicinity of 400 nm are shown.

At the tip of the endoscope (namely, the distal end of the scope body 2), an observation window is provided adjacent to the illumination window. The objective optical system 13 is mounted to the observation window, and reflected light and fluorescent light from the part 12 to be examined are incident thereon to form an image on the image sensor 5. The excitation wavelength cutoff filter 6 is placed in the objective optical system 13 so that the wavelength component of excitation light is blocked with respect to light incident on the objective optical system 13 and participating in image formation. Also, the excitation wavelength cutoff filter 6 may be constructed with a single plate or, as shown in FIG. 1, with a plurality of plates.

The excitation wavelength cutoff filter 6, as shown in FIG. 4, has the characteristics of blocking light in the transmission wavelength regions of the filters 7-Ex1 and 7-Ex2 of the excitation wavelength selective filter 7 and of transmitting fluorescent light of an auto-emission substance, such as collagen, contained in the visible region and fluorescent light of Alexa 680. In the wavelength regions of these two kinds of excitation light, the transmission characteristic of at least optical density (OD) 4 is required to completely block light, and therefore, the filter 6 must be constructed of a multilayer film of about 100-200 layers. Also, since fluorescence wavelengths of the auto-fluorescence substance, such as collagen, overlap a part of visible wavelengths, reflected light from the living body 12 of visible light can also be acquired by the characteristic of the excitation wavelength cutoff filter 6.

The film formation of at least 100 layers can be achieved by an ion film deposition technique and a sputtering process that have attained remarkable developments of apparatuses in recent years. Hence, it is possible that a plurality of kinds of excitation light with different wavelengths are blocked by the excitation wavelength cutoff filter 6 and only the fluorescent component of the auto-fluorescence substance, such as collagen, with a different wavelength and the fluorescent component of the fluorescent medicine Alexa 680 are transmitted to form an image on the image sensor 5. Further, the excitation wavelength cutoff filter 6 also has the characteristic of transmitting a part of light in the visible region, and thus visible reflected light containing the aspect information of the living body can be acquired.

A signal received by the image sensor 5 is input into the processor 3 through a signal line. The processor 3 has a preprocess circuit 15 conducting the amplitude of an image signal of the image sensor 5 and preprocessing such as white balance, an A/D conversion circuit 16, an image signal processing circuit 17 performing processing such as image enhancement, a D/A conversion circuit 18, and a filter control circuit 14.

The filter control circuit 14 controls the rotation drive of the excitation wavelength selective filter 7 and individual filters so as to synchronize fluorescent light imaged by the image sensor 5 or visible reflected light.

The image signal processing circuit 17 is constructed so that processing of the synthesis of a fluorescent image and a visible image is also performed and so that an image in which a lesion part is easily recognized and can be exactly diagnosed is provided to an observer (or a diagnostician).

The image signal output from the D/A conversion circuit 18 is input into the monitor 4. A fluorescent image and a visible image that are formed on the imaging surface of the image sensor 5 are displayed on the display surface of the monitor 4.

Generally, in NTSC of video format, the frame rate is 30 frames/sec. However, when compared with the intensity of reflected light in the visible region from the living body, the intensity of fluorescent light (including fluorescent light from the auto-fluorescence substance and fluorescent light from the fluorescent medicine) is as low as 10³-10⁴. Consequently, it is necessary to improve an S/N ratio in such a manner that the frame rate is lowered and the exposure time per frame is increased. However, when the frame rate is reduced to 3 frames/sec, images, such as low speed shot, are obtained and are hard for the observer (or the diagnostician) to observe.

The auto-fluorescence substance existing inside the living body has the absorption peak of light at a wavelength of 500 nm or less. For example, light of wavelength 405 nm is capable of efficiently exciting collagen and elastin that exist in the submucosa of the living tissue. It is practically avoided that the substance absorbs light of wavelengths more than 500 nm to emit fluorescent light. Thus, a substance having the absorption peak of light at any of wavelengths more than 500 nm is used as the fluorescent medicine introduced into the living body so that the fluorescent medicine is not excited during observation in which the auto-fluorescence substance is used. In this way, it is prevented that fluorescent light from the auto-fluorescence substance becomes a noise to obstruct the observation in which the fluorescent medicine is used.

The wavelength interval of excitation light relating to each fluorescent substance is properly provided, and thereby even when a wide wavelength region of light containing an absorption peak wavelength of light of the fluorescent substance is set as the excitation light, the fluorescent substance can be efficiently excited because the wavelength regions of the excitation light fail to overlap. Thus, the wavelengths of light exciting the fluorescent medicine and light exciting the auto-fluorescence substance are definitely separated, and thereby fluorescent light from the fluorescent medicine and the auto-fluorescence substance can be detected with good contrast and brightness.

In order to detect a plurality of different kinds of fluorescent light as in this embodiment, it is necessary to increase the exposure time per frame accordingly. For this, the frame rate must be lowered. Therefore, the fluorescence endoscope apparatus of Embodiment 1 is designed as described below. FIGS. 6A-6D and 7A-7D are characteristic diagrams of timing for acquiring the image of the fluorescence endoscope apparatus of Embodiment 1, showing timing charts of the screening mode and the minute investigation mode, respectively.

In the case where the fluorescence endoscope apparatus of this embodiment is used to carry out the observation and diagnosis inside the living body, the screening mode is first selected and the location where the lesion exists in the living body is searched and specified. In the screening mode, the frame rate is set to 30-10 frames/sec.

When the lesion part is specified, the mode is switched to the minute investigation mode and an image for making exact diagnosis of the lesion part is acquired. In the minute investigation mode, the frame rate is set to the value equivalent to the screening mode or less.

In the screening mode, the first filter 7-Ex1 exciting the auto-fluorescence substance, such as collagen, and the visible light filter 7-Vis, of the excitation wavelength selective filter, are used and, as shown in FIG. 6A, the living body 12 is irradiated in turn with illumination light from the light source device 1.

In this case, as shown in FIGS. 6B and 6C, when the living body 12 is irradiated with visible light in a predetermined wavelength region through the visible light filter 7-Vis from the light source device 1, light reflected by the living body 12 is chiefly observed, while when the living body 12 is irradiated with excitation light for exciting the auto-fluorescence substance, such as collagen, through the first filter 7-Ex1 from the light source device 1, auto-fluorescence from the auto-fluorescence substance existing in the living body is observed. Consequently, as shown in FIG. 6D, images of an object by reflected light from the living body 12 and by the auto-fluorescence from the auto-fluorescence substance, such as collagen, can be acquired in time series.

In the minute investigation mode, the first filter 7-Ex1 exciting the auto-fluorescence substance, such as collagen, the second filter 7-Ex2 exciting the fluorescent medicine Alexa Fluor 680, and the visible light filter 7-Vis, of the excitation wavelength selective filter, are used and, as shown in FIG. 7A, the living body 12 is irradiated in turn with illumination light from the light source device 1.

In this case, as shown in FIGS. 7B and 7C, when the living body 12 is irradiated with excitation light for exciting the auto-fluorescence substance, such as collagen, through the first filter 7-Ex1 from the light source device 1, auto-fluorescence from the auto-fluorescence substance existing in the living body is observed. When the living body 12 is irradiated with excitation light for exciting the fluorescent medicine Alexa Fluor 680 through the second filter 7-Ex2 from the light source device 1, fluorescent light emitted from the fluorescent medicine Alexa Fluor 680 peculiarly accumulated in the lesion part is observed. When the living body 12 is irradiated with visible light in a predetermined wavelength region through the visible light filter 7-Vis from the light source device 1, light reflected by the living body 12 is chiefly observed.

Hence, as shown in FIG. 7D, images of the object by reflected light from the living body 12, by auto-fluorescence from the auto-fluorescence substance, such as collagen, which is the first fluorescent light, and by fluorescent light from the fluorescent medicine Alexa Flour 680 which is the second fluorescent light can be acquired in time series by the image sensor 5.

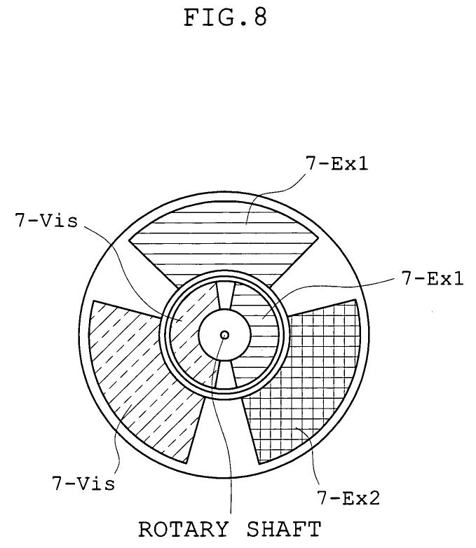

In the structure of the excitation wavelength selective filter 7, as shown in FIG. 8, the filter 7 may be placed on the optical path in such a way that it is separated into inner and outer circumferences for the screening and minute investigation modes. By doing so, complicated rotation control becomes unnecessary in the switching of the observation mode and thus a rotation driving device can be simplified.

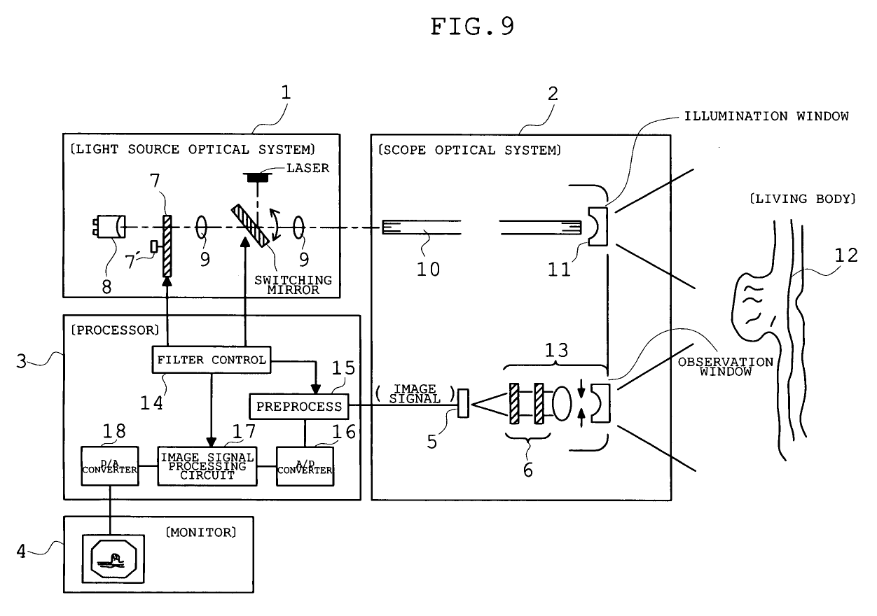

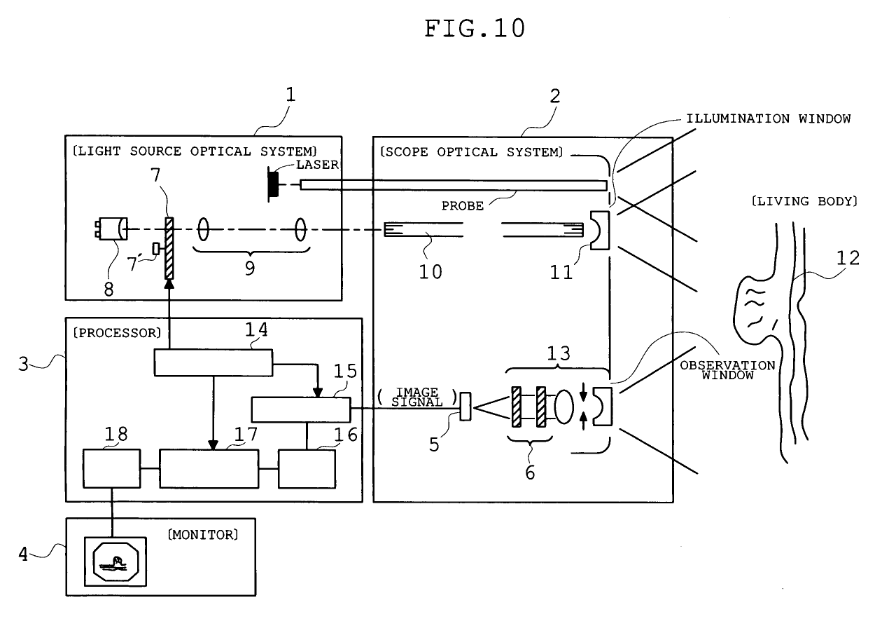

In the light source device 1, as shown in FIGS. 9 and 10, a semiconductor laser (LD) is used as a light source for excitation in addition to the lamp 8, and the optical system may be constructed so that the optical path can be switched (FIG. 9) or two kinds of light are used (FIG. 10). The semiconductor laser, which produces high output in a narrow wavelength region, is suitable for exciting the auto-fluorescence substance and pigment that emit faint fluorescent light. Also, in this case, the excitation wavelength selective filter is such as to block light in the regions excluding the visible light filter 7-Vis shown in FIG. 2.

According to the fluorescence endoscope apparatus constructed as mentioned above, its structure is simple, wavelengths of light irradiating on the light source side can be controlled in time series, and the imaging timing of different kinds of fluorescent light can be adjusted. In addition, since the wavelength interval of excitation light relating to each fluorescent substance is properly provided so that the number of a plurality of kinds of excitation light becomes equal to the number of detections of fluorescent light, the excitation wavelength cutoff filter is capable of having simple characteristics and the separation between the wavelengths of fluorescent light from the fluorescent substance and visible reflected light becomes possible.

Consequently, the light source device for excitation and the excitation wavelength cutoff filter can also be designed to have simple structures. In a video type endoscope in which the image sensor is placed at the distal end, the endoscope in which excitation can be created with light of a plurality of wavelengths and fluorescent light identical in number of kinds with excitation light is wavelength-separated and detected can be realized at a relatively low cost. Furthermore, as a result, a low-cost, distal-end video type endoscope is obtained in which ability for an ordinary visible-light observation is combined and a further improvement in diagnosis ability for the lesion, such as cancer, can be expected.

### Embodiment 2

Subsequently, Embodiment 2 of the endoscope apparatus of the present invention will be explained.

The endoscope apparatus of Embodiment 2 is the same as the endoscope apparatus of Embodiment 1 with the exception of the characteristics of the excitation wavelength selective filter in the minute investigation mode and the characteristics of the excitation wavelength cutoff filter placed in the objective optical system.

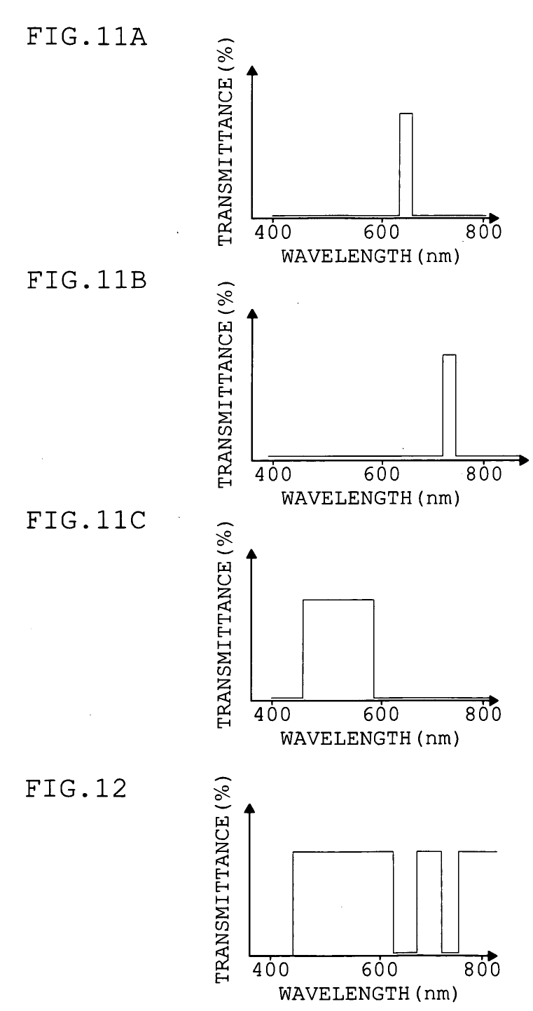

FIGS. 11A, 11B, and 11C are graphs showing spectral transmittance characteristics of the first excitation wavelength selective filter 7-Ex1, the second excitation wavelength selective filter 7-Ex2, and the visible light filter 7-Vis, respectively, used in the endoscope apparatus of Embodiment 2.

The first excitation wavelength selective filter 7-Ex1 excites Alexa 680 that is the fluorescent medicine emitting fluorescent light in the near infrared region and thus, as shown in FIG. 11A, has the transmittance characteristic of transmitting light in the vicinity of 680 nm that is the absorption peak wavelength of light of Alexa Flour 680. The second excitation light filter 7-Ex2 excites Alexa Flour 750 that is the fluorescent medicine likely emitting fluorescent light in the near infrared region and thus, as shown in FIG. 11B, has the transmittance characteristic of transmitting wavelengths in the vicinity of 750 nm that is the absorption peak wavelength of light of Alexa Flour 750.

The visible light filter 7-Vis acquires the aspect information of the living body from reflected light from the living body to reflect it in fluorescence information and thus, as shown in FIG. 11C, has the transmittance characteristic of transmitting wavelengths ranging from about 420 nm to about 650 nm in the visible region.

FIG. 12 is a graph showing the spectral transmittance characteristics of the excitation wavelength cutoff filter placed in the objective optical system used in the endoscope apparatus of Embodiment 2.

The excitation wavelength cutoff filter 6, as shown in FIG. 12, has the characteristics of blocking light in the transmission wavelength regions of the excitation wavelength selective filters 7-Ex1 and 7-Ex2 and of transmitting the fluorescent light of Alexa 680 and the fluorescent light of Alexa 750. In the wavelength regions of these two kinds of excitation light, the transmission characteristic of at least OD 4 is required to completely block light, and therefore, the filter 6 must be constructed of a multilayer film of about 100-200 layers. Also, the excitation wavelength cutoff filter 6 combines the characteristic of transmitting light in the wavelength region 420-650 nm.

As the light source device 1 used in the endoscope apparatus of Embodiment 2, the semiconductor laser may be used. In order to fabricate a band-pass filter blocking light in a wide wavelength region such as shown in FIGS. 11A and 11B, it is necessary to provide the multilayer film of more than about 200 layers, which is difficult to fabricate. When the semiconductor laser is used, the manufacture of the light source device can be facilitated and a high-function and lower-cost apparatus can be provided. Also, in this case, the excitation wavelength selective filter 7 is such as to block light in the regions excluding the visible light filter 7-Vis shown in FIG. 11.

According to the endoscope apparatus of Embodiment 2, only the fluorescent medicine emitting fluorescent light in the near infrared region having the tumor affinity is used and thereby function information as to a plurality of cancers can be obtained from a deep region in the submucosa of the living body. In addition, when the fluorescent medicine is prepared so that a plurality of kinds of substances to be detected, characterizing the lesion (for example, substances participating when the lesion deteriorates or when the lesion multiplies actively) are captured and combined, diagnosis accuracy relative to the lesion can be rapidly improved.

In this case, since the wavelength interval at the absorption peak of light of each fluorescent medicine is sufficiently provided, it is practically avoided that during observation in which one fluorescent medicine is used, another fluorescent medicine is excited. Thus, it can be prevented that fluorescent light from another fluorescent medicine becomes a noise to obstruct the observation.

Other structures, functions, and effects are almost the same as in the endoscope apparatus of Embodiment 1.

### Embodiment 3

Subsequently, Embodiment 3 of the fluorescence endoscope apparatus of the present invention will be explained.

The endoscope apparatus of Embodiment 3 is the same as the endoscope apparatus of Embodiment 2 with the exception of the characteristics of the excitation wavelength selective filter in the screening mode and the characteristics of the excitation wavelength cutoff filter placed in the objective optical system.

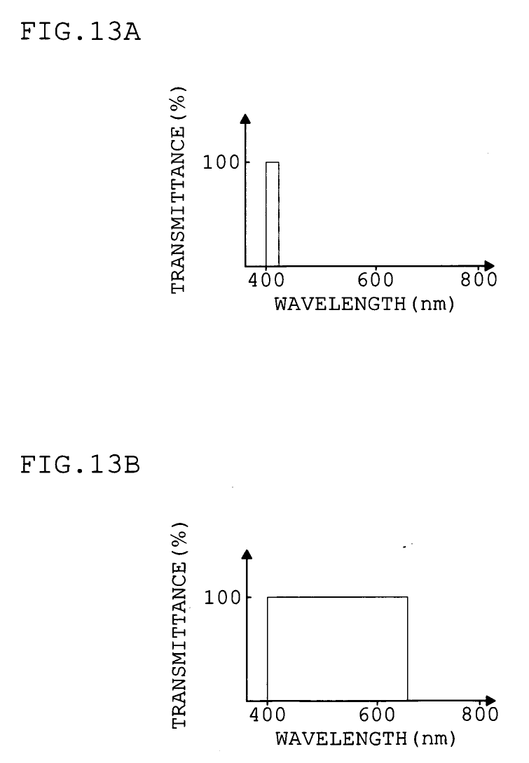

FIGS. 13A and 13B are graphs showing spectral transmittance characteristics of the first excitation wavelength selective filter 7-Ex1 and the visible light filter 7-Vis, respectively, in the screening mode, used in the endoscope apparatus of Embodiment 3.

In order to find the lesion as easily as possible, this embodiment is constructed so that in one of special image observations combined with common color image observations, an observation technique, called Narrow Band Imaging (hereinafter abbreviated to NBI), of irradiating the living tissue with band light in a narrow wavelength region and of imaging and observing reflected light from the living tissue is used and thereby the location and area of the lesion in the living body can be specified. The features of the NBI are as described below.

For example, in the case of light of short wavelengths such as blue light, a penetration depth into the living body is small. Consequently, when band light of short wavelengths in the narrow wavelength region is used in the NBI, the light of short wavelengths contains only information of the surface of the living tissue and its surroundings and is reflected, and hence an observation image specifying the surface of the living tissue can be obtained. On the other hand, for example, where light that the penetration depth into the living body is large, such as red light, is used, light of long wavelengths contains information of a deep portion of the living tissue and is reflected, and thus a state of the deep portion of the living tissue can be imaged.

The NBI is capable of providing a sharp image, for example, of the capillary of the mucosa layer without dispersing the pigment on the surface of the living body or without injecting a contrast medium, such as indocyanine green (ICG), around the tumor produced in the living tissue. The lesion tissue, such as the tumor, involves the multiplication of the capillary from the initial stage of its production. Therefore, as in the fluorescence endoscope apparatus of this embodiment, the common color image observation and the NBI are combined and thereby the location where the abnormality of the capillary is produced can be easily found.

In order to carry out the observation by the NBI, the first excitation wavelength selective filter 7-Ex1, as shown in FIG. 13A, has the transmittance characteristic of transmitting light in an extremely narrow wavelength region about 415 nm±15 nm. In order to carry out the color image observation by visible light, the visible light filter 7-Vis, as shown in FIG. 13B, has the transmittance characteristic of transmitting light in the wavelength region 400-650 nm.

Also, in this case, the characteristics of the excitation wavelength selective filer used in the minute investigation mode are the same as those shown in FIGS. 11A and 11B in Embodiment 2.

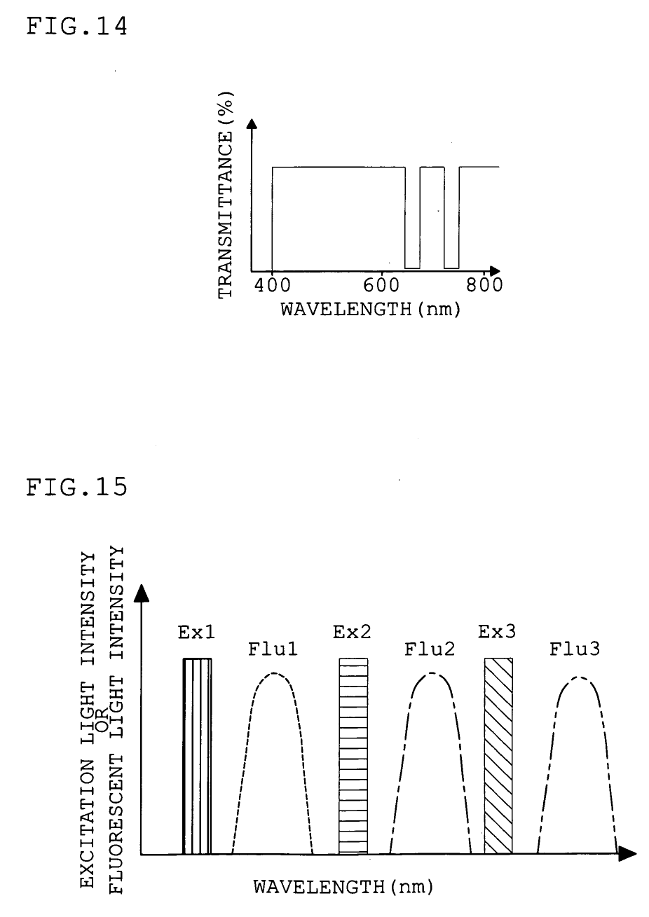

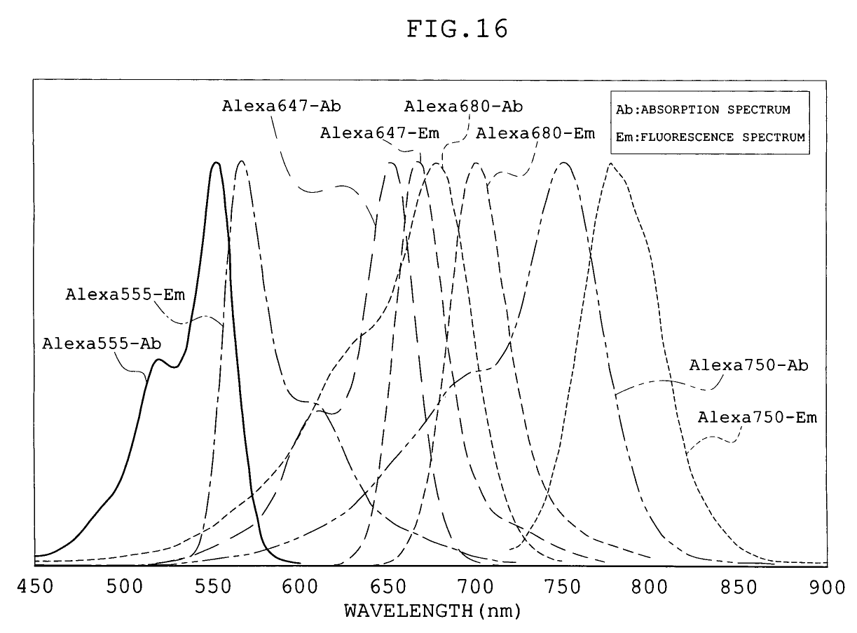

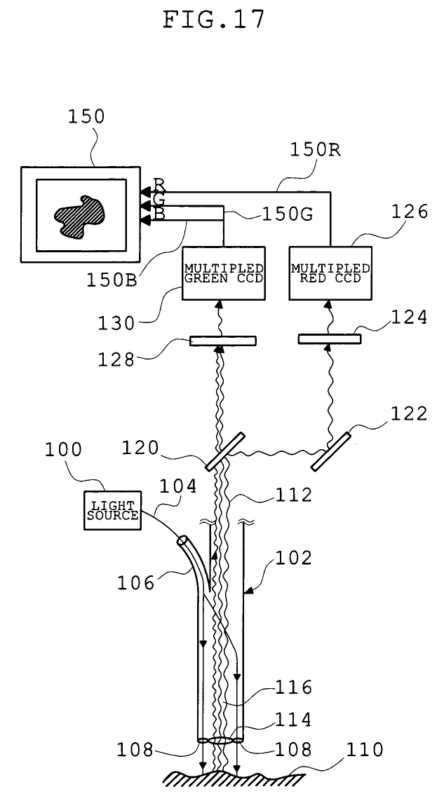

FIG. 14 is a graph showing the spectral transmittance characteristic of the excitation wavelength cutoff filter placed in the objective optical system used in the endoscope apparatus of Embodiment 3. The excitation wavelength cutoff filter 6, as shown in FIG. 14, has the characteristic of blocking excitation light in the wavelength region used in the minute investigation mode.

In the timing of the image acquirement, although omitted from the figure, the same drive as in the endoscope apparatus of Embodiment 1 is conducted.

### Embodiment 4

Subsequently, Embodiment 4 of the endoscope apparatus of the present invention will be explained.

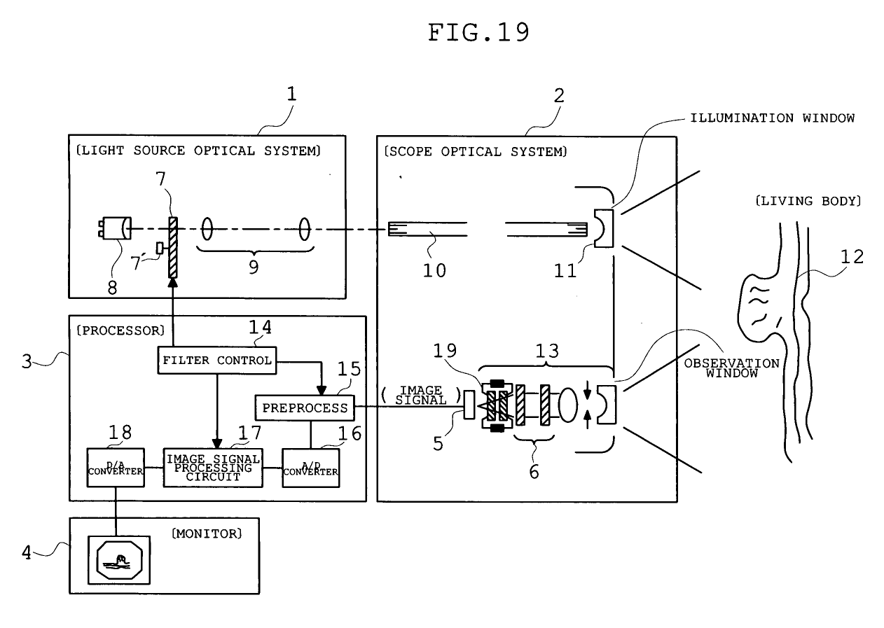

FIG. 19 is a block diagram showing the entire structure of the endoscope apparatus of Embodiment 4 in the present invention. In addition to the excitation wavelength cutoff filter used in the endoscope apparatus of each of Embodiments 1-3, the endoscope apparatus of Embodiment 4 is constructed so that a Fabry-Perot air gap variable tunable filter 19 used as a fluorescence wavelength selective filter splitting fluorescent light is placed in the objective optical system 13. According to the endoscope apparatus of Embodiment 4 constructed as mentioned above, it becomes possible to split the wavelength in a fluorescence wavelength region, much function information on cancer can be acquired, and the improvement of the diagnosis ability for cancer is expected.

Other structures, functions, and effects are almost the same as the endoscope apparatus of Embodiment 1.

### Embodiment 5

Subsequently, Embodiment 5 of the endoscope apparatus of the present invention will be explained.

As the image sensor 5 used in the endoscope apparatus of each of Embodiments 1-4, the endoscope apparatus of Embodiment 5 uses an image sensor, unlike a common monochromatic image sensor, in which a mosaic filter is placed immediately before a light-receiving element, not shown. The mosaic filter has the characteristic of blocking excitation light. According to the endoscope apparatus of Embodiment 5 constructed in this way, even when the object is irradiated with a plurality of kinds of excitation light on the light source side at the same time, the image sensor is capable of detecting the split of fluorescent light and the simplification of the light source device is feasible. Other structures, functions, and effects are almost the same as the endoscope apparatus of Embodiment 1.

Also, the fluorescent medicine used in the present invention is not limited to that used in the endoscope apparatus of each of Embodiments 1-4, and any substance with the absorption spectrum and the fluorescence spectrum in the near infrared wavelength region is satisfactory. The number of a plurality of kinds of excitation light, the number of detections of fluorescent light, and the kinds of fluorescent medicine are not limited to two kinds, but may be of three or more kinds.

What is claimed is:

1. An endoscope apparatus comprising:
a light source section emitting excitation light for exciting a fluorescent substance;
an excitation light supply section irradiating a living body with light emitted from the light source section; and
a fluorescence detection section provided with an imaging optical system for imaging fluorescent light emanating from the living body,
wherein the light source section has excitation wavelength selective means selecting a plurality of kinds of excitation light in different wavelength regions and the imaging optical system has an excitation wavelength cutoff filter blocking light of excitation wavelength selected by the excitation wavelength selective means so that the fluorescence detection section detects the number of kinds of fluorescent light in different wavelength region, identical with the number of kinds of excitation light in different wavelength regions, irradiated through the excitation light supply section.

2. An endoscope apparatus according to claim 1, wherein the light source section is constructed so that excitation light for an auto-fluorescence substance exciting the auto-fluorescence substance existing originally in the living body and excitation light for an administration fluorescent substance different in wavelength from the excitation light for the auto-fluorescence substance exciting a fluorescent substance administered inside the living body from an exterior are emitted through the excitation wavelength selective means.

3. An endoscope apparatus according to claim 1, wherein the excitation light supply section is constructed so that a plurality of fluorescent substances administered inside the living body from an exterior through the excitation wavelength selective means are excited in different wavelength regions.

4. An endoscope apparatus according to claim 3, wherein the light source section is constructed so that a plurality of kinds of fluorescent light in different wavelength regions, emitted from the light source section, have wavelengths more than 600 nm in a near infrared region.

5. An endoscope apparatus according to claim 1, wherein the fluorescence detection section is constructed so that fluorescent light in a predetermined wavelength region between adjacent excitation wavelength regions is detected with respect to a plurality of kinds of excitation light in different wavelength regions, emitted from the light source section.

6. An endoscope apparatus according to claim 1, wherein the fluorescence detection section is provided at a tip of a scope.

7. An endoscope apparatus according to claim 1, wherein the fluorescence detection section has a single image sensor.

8. An endoscope apparatus according to claim 1, wherein the excitation light supply section is constructed so that the living body is irradiated with a plurality of kinds of excitation light in different wavelength regions, each in different timing.

9. An endoscope apparatus according to claim 7, wherein the image sensor of the fluorescence detection section includes a monochrome CCD.

10. An endoscope apparatus according to claim 1, wherein an image sensor of the fluorescence detection section includes a color mosaic CCD.

11. An endoscope apparatus according to claim 1, wherein the light source section is provided with a light source for observations irradiating the living body with white light in different timing, together with a plurality of kinds of excitation light in different wavelength regions.

12. An endoscope apparatus according to claim 11, wherein the excitation wavelength cutoff filter has a characteristic of transmitting at least a part of light in a visible wavelength region.

13. An endoscope apparatus according to claim 1, having at least two kinds of image modes for observations and diagnoses of a screening mode and a minute investigation mode in which fluorescence detections are different in number.
